SEQUENCE LISTING (1 sequence)

SEQ ID NO: 1 - protein, 364 aa, Homo sapiens
ENSDDYDLMYVNLDNEIDNGLHPTEDPTPCACGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS

(19) United States
(12) Patent Application Publication
(10) Pub. No.: US 2025/0283894 A1
(43) Pub. Date: Sep. 11, 2025

(54) PTX3 BIOMARKER FOR REDUCING ANTIBIOTIC TREATMENT IN NEWBORNS

(71) Applicants: bioMérieux, Marcy-l'Etoile (FR); Hospices Civils de Lyon, Lyon (FR); Universite Claude Bernard Lyon 1, Villeurbanne (FR)

(72) Inventors: Marine Butin, Chassieu (FR); Sylvie Pons, Saint-Genis-les-Ollieres (FR); Sophie Assant-Trouillet, Reyrieux (FR); Karen Brengel Pesce, Les Abrets en Dauphine (FR); Fabien Subtil, Lyon (FR)

(21) Appl. No.: 18/855,684

(22) PCT Filed: Apr. 7, 2023
(86) PCT No.: PCT/FR2023/050511

§ 371 Date: Oct. 10, 2024

(30) Foreign Application Priority Data
Apr. 11, 2022 (FR) 2203313
Apr. 11, 2022 (FR) 2203314

(51) Int. Cl. G01N 33/68

(52) U.S. Cl. CPC G01N 33/6893; G01N 2333/4703; G01N 2800/26; G01N 2800/38; G01N 2800/52

(57) ABSTRACT

The invention relates to an in vitro method for determining whether a newborn patient has late-onset neonatal sepsis, which comprises determining the PTX-3 protein concentration in a biological sample previously obtained from said patient.

### STATEMENT REGARDING ELECTRONIC FILING OF A SEQUENCE LISTING

A Sequence Listing in XML format, entitled 1716-2_ST26.xml, 2,080 bytes in size, generated Jan. 13, 2025, and filed herewith, is hereby incorporated by reference into the specification for its disclosures.

### TECHNICAL FIELD

The invention relates to an in vitro method for determining whether a newborn patient has late-onset neonatal sepsis, comprising determining the protein concentration of PTX-3 in a biological sample previously obtained from said patient.

### PRIOR ART

Sepsis is one of the major causes of morbidity and mortality in newborns. Neonatal sepsis is an invasive, usually bacterial, infection that occurs during the neonatal period. There are two categories of neonatal sepsis, namely early-onset neonatal sepsis, which usually appears before the first three days of life of the newborn, or late-onset neonatal sepsis, which appears on the fourth day or later. Neonatal sepsis is said to be late-onset as opposed to early-onset infections which are usually transmitted from the mother to the newborn during pregnancy or childbirth. The symptoms are many, non-specific and complex, and diagnosis is based on the results of laboratory blood culture. Diagnosis by blood culture risks false negatives and the volume of blood needed for an optimal culture can sometimes not be taken from very-low-weight newborns. In addition, it generally takes up to 48 hours to receive the results of the blood culture.

Due to the rapid progression of sepsis and its high mortality rate, rapid empirical antibiotic therapy is generally administered to the newborn on arrival thereof in the neonatal unit in the event of suspicion of sepsis and while awaiting the results of the blood culture. Thus, a certain proportion of patients receive antibiotic treatment when it is ultimately not necessary.

However, the systematic use of antibiotic therapy in newborns, besides contributing to the emergence of multi-resistant bacteria, can also have deleterious effects, in particular mortality during hospitalization (Ting et al. 2016. JAMA pediatrics, 170(12):1181-1187) or, over the medium/long term, a disruption in the microbiota which may be involved in the occurrence of later pathologies such as respiratory difficulties (Kummeling I. et al. Pediatrics, 2007. 119(1): e225-231).

It is therefore imperative to develop rapid and reliable diagnostic tests for early determination of the absence of late-onset neonatal sepsis in newborns in order to minimize the use of antibiotic therapy, especially prolonged use in newborns who ultimately turn out not to have an infection. A rapid diagnosis would thus make it possible to determine whether treatment with antibiotics needs to be initiated or continued when it has been prescribed and administered in the event of suspicion of late-onset neonatal sepsis and thus to maintain antibiotic therapy solely for the newborns actually exhibiting late-onset neonatal sepsis.

A certain number of biomarkers have been studied for the diagnosis of sepsis in newborns. Procalcitonin (PCT), an early inflammation factor used as a biomarker for bacterial infections in adults, has frequently been used as a diagnostic marker for neonatal sepsis. Nevertheless, its clinical use for diagnosing neonatal sepsis is debated (Pontrelli G. et al. BMC Infect Dis. 2017; 17: 302; Eschborn S and Weitkamp J-H, Journal of Perinatology, 2019; 39(7): 893-903) and this marker has to be used in combination with other clinical markers and laboratory data in order to evaluate the need for continuing antibiotic therapy.

There is therefore a need to identify a biomarker that makes possible rapid and reliable diagnosis of late-onset neonatal sepsis, making it possible to precisely rule out the case of neonatal sepsis but also avoid false positives and avoid or reduce the exposure to antibiotics in newborns.

Pentraxin 3 (PTX-3) is a glycoprotein expressed by endothelial cells and mononuclear phagocytes. An elevated level of PTX-3 has been shown to be an indicator of meningococcal shock in infants. However, the role of PTX3 in the diagnosis of neonatal sepsis has been subject to only little study (Sharma et al. 2018, J Matern Fetal Neonatal Med. 2018 June; 31(12): 1646-1659; Baumert M. et al. Biomed Res Int, 2021 Jun. 30; 2021:6638622). Thus, rigorous, larger-scale studies are needed for precisely evaluating the efficacy of this biomarker for its clinical use for reducing antibiotic therapy in newborns and in the diagnosis of neonatal sepsis.

### SUMMARY

The present disclosure improves the situation. In the present application, the inventors have conducted a large-scale prospective and multicenter study in two neonatal intensive care and resuscitation units to evaluate the reliability of different biomarkers as tools for reducing antibiotic therapy in newborns, for example in the stopping of treatments with antibiotics initially administered following a suspicion of infection, or for diagnosing the absence of late-onset neonatal sepsis. The inventors have shown, surprisingly, that, among these biomarkers and contrary to IL-6, the determination of the protein concentration of PTX3 makes it possible to rule out the presence of late-onset neonatal sepsis. In addition, determination of the protein concentration of PTX3 advantageously combined with determination of the protein concentration of NGAL and optionally gelsolin makes possible the avoidance or early reduction of antibiotic therapy in more than half of patients diagnosed as not having late-onset neonatal sepsis, for example following blood culture results or expert opinions. This performance is maintained when this biomarker is combined for example with other biomarkers such as IL-10, IL-6, calprotectin, LBP, PCT, sCD14 or IP10.

The PTX3 biomarker, used alone or in combination, thus finds a very particular application in patients hospitalized in the neonatal unit as a tool in the early stopping of antibiotic treatments initially prescribed following suspicion of late-onset neonatal sepsis.

The present invention relates to an in vitro method for determining whether a treatment with an antibiotic is able to be stopped in a newborn patient who possibly has late-onset neonatal sepsis and has previously received treatment with the antibiotic, said method comprising determining the protein concentration of PTX-3 in a biological sample previously obtained from said patient and comparing the concentration thus determined with a reference threshold value, characterized in that a protein concentration of PTX-3 lower than said reference threshold value indicates that the treatment with the antibiotic is able to be stopped and that the patient does not have late-onset neonatal sepsis.

According to a preferred embodiment, the method also comprises determining the protein concentration of NGAL in a biological sample previously obtained from said patient and comparing the concentration thus determined with a reference threshold value. According to this embodiment, protein concentrations of PTX-3 and NGAL lower than the reference threshold values indicate that the treatment with the antibiotic is able to be stopped.

According to another preferred embodiment, the method also comprises determining the protein concentrations of NGAL and gelsolin in a biological sample previously obtained from said patient and comparing the concentrations thus determined with reference threshold values. According to this embodiment, protein concentrations of PTX-3, NGAL and gelsolin lower than the reference threshold values indicate that the treatment with the antibiotic is able to be stopped.

Preferably, the protein concentration of PTX-3 is determined by an immunoenzymatic ELISA method, preferably a microfluidic ELISA method. In a particular embodiment, said biological sample is a biological blood, plasma or serum sample, preferably a biological serum sample, and preferably the volume of the biological sample previously obtained from the patient is less than 50 μL, preferably less than 20 μL, and more preferably less than 5 μL.

In a particular embodiment, the reference threshold value for the protein concentration of PTX-3 is less than 3000 pg/mL, preferably less than 2000 pg/mL, preferably less than 1300 pg/mL and more preferably between 1000 and 1300 pg/mL, for example 1266 pg/mL.

In another particular embodiment, the method according to the present invention may comprise determining the concentration of at least one other protein chosen from: IL-6, IL-10, PCT, IP10, sCD14, LBP, NGAL, calprotectin and gelsolin, said method being characterized in that concentrations of the protein PTX-3 and of at least one of said other proteins lower than corresponding reference threshold values indicate that the patient does not have late-onset neonatal sepsis and/or that the treatment with the antibiotic is able to be stopped. Preferably, according to this embodiment, said method comprises determining the concentration of the protein NGAL or concentrations of the proteins NGAL and gelsolin, said method being characterized in that concentrations of the proteins PTX3, NGAL and optionally gelsolin lower than corresponding reference threshold values indicate that the patient does not have late-onset neonatal sepsis and/or that the treatment with the antibiotic is able to be stopped.

According to a variant of this embodiment, the method according to the present invention may comprise determining the concentration of at least two other proteins chosen from: IL-6, IL-10, PCT, IP10, sCD14, LBP, NGAL, calprotectin and gelsolin, and more preferably chosen from IL-10, NGAL and gelsolin.

In another particular embodiment, the method according to the present invention is characterized in that only the concentration of PTX-3 is determined.

In a preferred embodiment, the biological sample is previously obtained from a patient exhibiting at least one clinical sign associated with a late-onset neonatal sepsis syndrome, preferably chosen from: a fever≥38° C., tachycardia≥160/min, capillary refill time (CRT)≥3 seconds, gray and/or pale complexion, bradycardia and apnea syndrome, increased ventilatory assistance and/or increase in FiO₂, abdominal bloating, rectal bleeding, hypotonia, lethargy, convulsions absent another obvious cause, skin rash or inflammation at the site of puncture of the central venous catheter, or a combination of these signs.

In another aspect, the present invention relates to an in vitro method for determining whether a newborn patient has late-onset neonatal sepsis, said method comprising determining the protein concentration of PTX-3 in a biological sample from said patient and comparing the concentration thus determined with a reference threshold value, characterized in that a concentration of PTX-3 greater than the reference threshold value indicates that the patient has late-onset neonatal sepsis.

In a final aspect, the present invention relates to an antibiotic for use thereof in the treatment of late-onset neonatal sepsis in a newborn patient, characterized in that said antibiotic is administered to said patient previously evaluated as having late-onset neonatal sepsis by the method as described above, said antibiotic preferably being chosen from amoxicillin, gentamicin and cefotaxime.

### BRIEF DESCRIPTION OF THE DRAWINGS

FIG. 1 shows a diagram of the organization of the monitoring of the study.

FIG. 2 shows an ROC curve and AUC of clinical signs.

FIG. 3 shows the ROC curves and corresponding AUCs for PTX3 biomarkers, and also for the combination of PTX3/NGAL and PTX3/NGAL/gelsolin biomarkers.

### DESCRIPTION OF THE EMBODIMENTS

In order to determine whether a patient has late-onset neonatal sepsis and to evaluate whether treatment of the patient with antibiotics should be administered or stopped, the inventors have shown, by carrying out a large-scale study on a large cohort of patients, that determination of the protein concentration of PTX3 in a biological sample from the patient compared to a reference threshold value makes it possible to evaluate whether said patient has late-onset neonatal sepsis. This thus makes it possible to limit antibiotic therapy in a patient who does not need it.

The present application relates to an in vitro method for determining whether a newborn patient has late-onset neonatal sepsis, said method comprising determining the protein concentration of PTX3 in a biological sample previously obtained from said patient and comparing the concentration thus determined with a reference threshold value, characterized in that a protein concentration of PTX3 lower than the reference threshold value indicates that said patient does not have late-onset neonatal sepsis.

The present application also relates to an in vitro method for determining whether a treatment with an antibiotic is able to be stopped in a newborn patient who possibly has late-onset neonatal sepsis and has previously received treatment with the antibiotic,
said method comprising determining the protein concentration of PTX-3 in a biological sample previously obtained from said patient and comparing the concentration thus determined with a reference threshold value,
characterized in that a protein concentration of PTX-3 lower than said reference threshold value indicates that the treatment with the antibiotic is able to be stopped and that said patient does not have late-onset neonatal sepsis.

Neonatal sepsis is an invasive, usually bacterial, infection that occurs during the neonatal period. Symptoms are many, non-specific and include decreased spontaneous activity, less vigorous suction, apnea, bradycardia, thermal instability, respiratory distress, vomiting, diarrhea, abdominal distension, nervousness, convulsions, jaundice or any other abnormal symptom. Neonatal sepsis can be categorized into two categories, early-onset neonatal sepsis, which usually appears before the first three days of life of the newborn, or late-onset neonatal sepsis, which appears on the fourth day or later (Pontrelli G. et al. BMC Infect Dis. 2017; 17: 302; Sharma et al. 2018, J. Matern Fetal Neonatal Med. 2018 June; 31(12):1646-1659).

As explained above, late-onset neonatal sepsis is usually contracted from the environment (neonatal nosocomial infections) and occurs after 3 days of life, as opposed to early-onset neonatal sepsis which is an infection generally transmitted from the mother to the newborn during pregnancy or childbirth (Sharma et al. 2018, J Matern Fetal Neonatal Med. 2018 June; 31(12):1646-1659). Staphylococci are responsible for 30 to 60% of late-onset infections and are most often due to intravascular devices (especially central vascular catheters). E. coli bacteria are also increasingly responsible for late-onset sepsis, especially in very low birth weight infants. Non-limiting examples of organisms that may cause neonatal sepsis include coagulase-negative staphylococci, including S. epidermidis, S. haemolyticus, S. hominis, S. warneri, S. saprophyticus, S. cohnii, and S. capitis, group B streptococci, Staphylococcus aureus, Enterococcus fecalis and E. faecium, Listeria monocytogenes, Escherichia coli, P. aeruginosa, Haemophilus influenzae, Streptococcus bovis, α-hemolytic streptococci, Streptococcus pneumoniae, Neisseria meningitides and N. gonorrhoeae.

Newborns suspected of having late-onset neonatal sepsis, in particular exhibiting one of the clinical signs suggestive of late-onset neonatal sepsis, are treated with antibiotic therapy. Specifically, as elaborated above, in the neonatal unit, for the time needed for the blood culture results to be available for confirming or not confirming the presence or absence of late-onset neonatal sepsis, which in practice is 48 h, the newborns are treated with antibiotics. The antibiotic that may be administered to the newborn patient can include, by way of example, one of the following antibiotics: amoxicillin, amikacin, aztreonam, chloramphenicol, ceftazidime, clindamycin, cefalotin, ciprofloxacin, colistin, cefotetan, cefotaxime, erythromycin, fusidic acid, fosfomycin, cefoxitin, furans, gentamicin, imipenem, kanamycin, lincomycin, cefamandole, minocycline, latamoxef, metronidazole, nalidixic acid, netilmicin, oxacillin, benzylpenicillin, pefloxacin, piperacillin, pristinamycin, rifampicin, spiramycin, sulfamides, streptomycin, trimethoprim, sulfamethoxazole, tetracycline, teicoplanin, ticarcillin, tobramycin, trimethoprim, vancomycin, preferably amoxicillin, gentamicin or cefotaxime. The antibiotics can be adapted according to the antibiogram and the location of the infection.

The inventors have shown that pentraxin 3 (PTX3) is a biomarker which makes it possible to reliably and rapidly evaluate the absence of late-onset neonatal sepsis in the newborn and/or whether antibiotic treatment is able to be initiated or stopped in said newborn.

In humans, the PTX3 gene, also called TSG-14 or TNFAIP5 (Gene ID: 5806 updated 20 Mar. 2022) codes for a member of the pentraxin family of proteins, PTX3 (UniProtKB-P26022, amended 23 Feb. 2022) comprising a 17-amino-acid signal peptide. The protein without signal peptide comprises the sequence SEQ ID NO: 1. PTX3 is a protein involved in immunity and also in inflammation by enabling the recognition of pathogenic molecules. The expression of PTX3 is induced by inflammatory cytokines in response to inflammatory stimuli in several types of mesenchymal and epithelial cells, especially endothelial cells and mononuclear phagocytes. The protein promotes fibrocyte differentiation and is involved in the regulation of inflammation and complement activation. It also plays a role in angiogenesis and tissue remodeling.

Determining the protein concentration of PTX3 in a biological sample previously taken from a newborn and comparing it to a reference threshold value makes it is possible to determine whether the newborn has late-onset neonatal sepsis and/or whether antibiotic treatment is able to be initiated or stopped, and preferably stopped, in said newborn.

The protein concentration of the biomarker in a biological sample according to the present disclosure, in particular of PTX3, can be determined by any appropriate method known to those skilled in the art. The protein concentration can be measured, for example, by semiquantitative Western blot, enzyme-labeled immunoassays, such as enzyme-linked immunosorbent assay (ELISA) methods, biotin/avidin-type assays, radioimmunoassays, immunoelectrophoresis, mass spectrometry, or immunoprecipitation or by protein or antibody arrays.

In a preferred embodiment, the protein concentration is determined by an ELISA method.

The ELISA method is a solid-phase enzymatic immunological test. This biochemical analysis technique falls within the more general framework of immunoenzymatic detection techniques (or EIA, for enzyme immunoassays), in which the recognition of an antigen under study by a specific antibody is monitored by virtue of an enzyme-catalyzed reaction and can generate the emission of a signal by a chromogenic or fluorogenic substrate. For this, the antigen is recognized by an antibody covalently coupled to an enzyme. The attachment of the labeled molecule will result, after the use of a chromogenic or fluorogenic substrate of the enzyme bound to the antibody, in the emission of a colored or fluorescent signal. The technique uses one or two antibodies. To facilitate the implementation of the technique, in particular in the case of a large number of samples to be analyzed, the antibody specific for the sought antigen or the antigen recognized by the sought antibody is bound to the support used which is then covered with it, hence the name immunosorbent technique. Several variations of operating protocols exist, making it possible to increase the specificity or sensitivity of recognition of the antigen (indirect, sandwich or competitive ELISA).

The antibodies or fragments thereof binding to the antigen used for the detection of the protein and the measurement of the protein concentration are the antibodies or fragments binding to the antigen binding specifically to the proteins of the present application (e.g. PTX3). The antibodies suitable for the present method can be chosen from among the antibodies known in the prior art.

The term “antibody” as used herein includes monoclonal antibodies, polyclonal antibodies and chimeric antibodies. The antibody can be obtained from recombinant sources and/or be produced by transgenic animals. The term “antibody fragment” as used herein is intended to include Fab, Fab′, F(ab′)2, scFv, dsFv, ds-scFv, dimers, minibodies, diabodies and multimers thereof and bispecific antibody fragments. The antibodies can be fragmented using conventional techniques. For example, F(ab′)2 fragments can be generated by treating the antibody with pepsin. The resulting F(ab′)2 fragment can be treated in order to reduce the disulfide bridges so as to produce Fab′ fragments. Digestion by papain can lead to the formation of Fab fragments. The Fab, Fab′ and F(ab′)2 fragments, scFv, dsFv, ds-scFv, dimers, minibodies, diabodies, bispecific antibody fragments and other fragments can also be synthesized by recombinant techniques.

Antibodies having specificity for one of the proteins described in the present application, such as PTX3, NGAL, IL-10, IL-6, calprotectin, LBP, PCT, sCD14, IP10 or gelsolin, can be prepared by conventional methods. A mammal (for example, a mouse, hamster, or rabbit) can be immunized with an immunogenic form of the peptide that causes an antibody response in the mammal. Techniques for imparting immunogenicity to a peptide include conjugation to carriers or other techniques well known in the art. For example, the peptide can be administered in the presence of an adjuvant. The progression of immunization can be monitored by the detection of antibody titers in plasma or serum. Standard ELISA procedures or other immunoassay procedures can be used with the immunogen as antigen for evaluating the antibody levels. After immunization, antisera can be obtained and, if desired, polyclonal antibodies can be isolated from the sera.

To produce monoclonal antibodies, antibody-producing cells (lymphocytes) can be taken from an immunized animal and fused with myeloma cells by standard somatic cell fusion procedures, thus immortalizing these cells and producing hybridoma cells. Such techniques are well known in the art (for example, the hybridoma technique originally developed by Kohler and Milstein (Nature 256:495-497 (1975)) and also other techniques such as the human B cell hybridoma technique (Kozbor et al., Immunol. Today 4:72 (1983)), the EBV hybridoma technique for producing human monoclonal antibodies (Cole et al., Methods Enzymol, 121:140-67 (1986)), and the screening of combinatorial antibody libraries (Huse et al., Science 246:1275 (1989)). Hybridoma cells can be immunochemically screened for the production of antibodies reacting specifically with the peptide and monoclonal antibodies can be isolated.

Alternatively, the antibodies may be commercial antibodies referenced for example in antibody databases such as the online database Antibodypedia, (Kiermer V. (2008) “Antibodypedia—A web portal to share antibody validation data” Nature methods. 5: 860).

In one embodiment of the present application, the antibodies or antibody fragments used in the ELISA methods which bind to proteins (e.g. PTX3) are labeled with a detectable label. The label is preferably capable of producing, directly or indirectly, a detectable signal. For example, the label may be radiopaque or a radioisotope, such as 3H, 14C, 32P, 35S, 123I, 125I, or 131I; a fluorescent compound (fluorophore) or chemiluminescent compound (chromophore), such as fluorescein isothiocyanate, rhodamine, or luciferin; an enzyme, such as alkaline phosphatase, beta-galactosidase, or peroxidase; an imaging agent; or a metal ion. In another embodiment, the detectable signal is indirectly detectable. For example, a labeled secondary antibody can be used to detect the protein of interest.

In newborns, it is difficult to obtain a large volume of biological sample due to technical constraints and legal constraints (Jardé law). Thus, in a preferred embodiment, in order to limit the volume of sample taken, the protein concentration of PTX3 is determined by a microfluidic ELISA method (Lee et al., “Microfluidic enzyme-linked immunosorbent assay technology,” Adv. CHn. Chem. 42: 255-259 (2006)). An example of an automatic microfluidic platform that can be used in the context of the present disclosure is described in Aldo P. et al. American Journal of Reproductive Immunology 75 (2016):678-693.

The biological sample previously taken from a patient may be a blood, plasma, serum or urine sample, preferably a blood, plasma or serum sample, and more preferentially a serum sample.

In a preferred embodiment, the volume of the biological sample previously collected from the patient is less than 50 μL, preferably less than 20 μL, more preferentially less than 5 μL, such as for example 3 μL.

The patient from whom the sample was previously taken is a newborn.

The term “newborn” denotes an infant within the first 28 days after birth. In certain embodiments, the newborn may be within the first 14 days of birth. Preferably, the patient is a newborn older than 3 days, preferably older than 7 days. A newborn evaluated in the context of the present application may be premature or full-term, for example, more particularly, a premature newborn weighing less than 1500 g.

According to the method of the present application, said patient is a newborn suspected of having late-onset neonatal sepsis. As mentioned above, it is commonly accepted that late-onset neonatal sepsis is sepsis occurring after 72 hours following birth.

According to one particular embodiment, the patient is a newborn suspected of having late-onset neonatal sepsis and who is treated with an antibiotic following said suspicion.

Clinical signs that might suggest late-onset neonatal sepsis are a worsening of general signs such as fever with a temperature above 38° C., hypothermia with a temperature below 36° C., worsening of respiratory signs such as respiratory distress, for example grunting, nasal flaring or signs of retraction, tachypnea with respiratory rate greater than 60/min and apnea, worsening of hemodynamic signs such as tachycardia greater than 160 bpm or bradycardia less than 80 bpm, signs of shock such as increased capillary refill time, pallor, arterial hypotension, oliguria, worsening of neurological signs such as drowsiness, irritability, hypotonia or convulsions, or worsening of digestive signs such as refusal to drink or vomiting.

In particular, said patient exhibits at least one of the clinical signs associated with late-onset neonatal sepsis, preferably chosen from: a fever≥38° C., tachycardia≥160/min, capillary refill time (CRT)≥3 seconds, gray and/or pale complexion, bradycardia and apnea syndrome increased ventilatory assistance and/or increase in FiO₂, abdominal bloating, rectal bleeding, hypotonia, lethargy, convulsions absent another obvious cause, skin rash or inflammation at the site of puncture of the central venous catheter, or a combination of these signs.

According to the present disclosure, the protein concentration of PTX3 in a biological sample previously taken from said patient is then compared to a reference threshold value. Comparing the protein concentration of PTX3 to a reference threshold value quickly and reliably indicates whether or not a patient has late-onset neonatal sepsis. It can thus make it possible to determine whether the treatment with an antibiotic should be initiated or stopped in the case where the latter has been administered preventively to the patient when said sepsis is suspected.

According to the present disclosure, a protein concentration of the PTX3 in a patient biological sample lower than a reference threshold value indicates that the patient does not have late-onset neonatal sepsis and preferably that the treatment with the antibiotic is able to be stopped. On the other hand, a protein concentration of the PTX3 in a patient biological sample greater than a reference threshold value indicates that the patient has late-onset neonatal sepsis and preferably indicates that the treatment with the antibiotic should be continued.

The term “reference threshold value”, as used herein, refers to a reference value, which may be predetermined, specifying, for example, a confidence interval or a threshold value for evaluating data obtained from a biological sample previously taken from a newborn patient and for diagnosing neonatal sepsis. The reference threshold value can also be derived from the protein concentration of one or more biomarkers, preferably from the corresponding protein concentration of the PTX3 in a “control” biological sample, for example a negative (uninfected) control or a positive (infected) control. The reference threshold value may be based on a large number of biological samples, such as for example from a population of subjects from the chronological age-matched group, or based on a pool of samples including or excluding the sample to be tested. In one particular embodiment, the reference threshold value according to the method of the present application may be obtained from one or more subjects not suffering from sepsis (that is to say negative control subjects). A subject is considered not to have sepsis if they have not been diagnosed as having sepsis, typically performed by blood culture or expert opinion.

The reference threshold value can also be obtained by determining the sensitivity and optimum specificity using an ROC (Receiver Operating Characteristic) curve based on experimental data. For example, as illustrated in the examples of the present application, after having determined the protein concentration of the biomarker (e.g. PTX3) in a reference group, algorithmic analysis can be used for the statistical processing of the values measured in the samples to be tested, and thus obtain a classification standard that has significance for the classification of the samples. In one particular embodiment, an analysis of the ROC with the protein concentration of the biomarker (e.g. PTX3) offering the greatest specificity for a sensitivity of at least 0.895 was chosen as the reference threshold value, and as described in the examples of the application. This algorithmic method is preferably carried out using a computer. Existing software or systems in the art can be used for plotting the ROC curve, such as: MedCalc 9.2.0.1, medical statistical software, SPSS 9.0, ROCPOWER.SAS, DESIGNROC.FOR, MULTIREADER POWER.SAS, CREATE-ROC.SAS, GB STAT V10.0 (Dynamic Microsystems, Inc. Silver Spring, Md., USA), etc.

In a preferred embodiment, according to the method of the present disclosure, the reference threshold value for the protein concentration of PTX3 is less than 3000 pg/mL, preferably less than 2000 pg/mL, preferably less than 1300 pg/mL and more preferably between 1000 and 1300 pg/mL, such as for example 1266 pg/mL.

In a preferred embodiment, according to the method of the present disclosure, the reference threshold value for the protein concentration of PTX3 is from 1000 to 3000 pg/mL, preferably from 1000 to 2000 pg/mL and more preferably from 1000 to 1300 pg/mL.

A protein concentration of the PTX3 in a biological sample previously obtained from said patient greater than a reference threshold value as described above indicates that the patient has late-onset neonatal sepsis. On the other hand, a protein concentration of the PTX3 in a biological sample previously obtained from said patient lower than said reference threshold value indicates that the patient does not have late-onset neonatal sepsis and preferably indicates that the treatment with the antibiotics is able to not be initiated, or be stopped if it has been prescribed.

The inventors in the present application have shown that it was possible to identify a threshold value of protein concentration of PTX3 making it possible to avoid or stop antibiotic therapy in patients who are ultimately not infected. Thus, in one particular embodiment, only the protein concentration of PTX3 is determined.

In another particular embodiment, the method according to the present disclosure may additionally comprise determining the protein concentration of at least one other protein chosen from: IL-6, IL-10, procalcitonin (PCT), IP10, sCD14, LBP, NGAL, calprotectin and gelsolin, preferably NGAL and optionally gelsolin, and comparing the concentrations thus determined with corresponding reference threshold values. According to this embodiment, concentrations of said proteins lower than corresponding reference threshold values indicate that the patient does not have late-onset neonatal sepsis. The reference threshold values are as defined below.

According to a variant of this embodiment, the method according to the present disclosure may comprise determining the protein concentration of at least two other proteins chosen from: IL-6, IL-10, PCT, IP10, sCD14, LBP, NGAL, calprotectin and gelsolin, and more preferably chosen from IL-10, NGAL and gelsolin, and comparing the concentrations thus determined with corresponding reference threshold values.

According to a preferred embodiment, the method according to the present application comprises determining the protein concentration of PTX3 and NGAL and comparing the concentrations thus determined with corresponding reference threshold values. According to this embodiment, concentrations of said proteins lower than corresponding reference threshold values indicate that the patient does not have late-onset neonatal sepsis and/or that the antibiotic treatment is able to be stopped.

According to another preferred embodiment, the method according to the present application comprises determining the protein concentration of PTX3, NGAL and gelsolin and comparing the concentrations thus determined with corresponding reference threshold values. According to this embodiment, concentrations of said proteins lower than corresponding reference threshold values indicate that the patient does not have late-onset neonatal sepsis and/or that the antibiotic treatment is able to be stopped.

In humans, interleukin 6 (IL6), also known as IFNB2, BSF-2, CDF, BSF2, HGF or HSF is a cytokine composed of 212 amino acids (UniProKB-P05231, updated 23 Feb. 2022) comprising a 29-amino-acid signal peptide encoded by the IL-6 gene (Gene ID: 3569, updated 27 Mar. 2022). IL-6 is produced by B and T lymphocytes and also monocytes, endothelial cells and fibroblasts and has a wide variety of biological functions in immunity, tissue regeneration and metabolism. IL6 binds to IL6R, and then the complex associates with the IL6ST/gp130 signaling subunit in order to trigger the IL6 intracellular signaling pathway. Advantageously, the reference threshold value of the protein concentration of IL6 is 15 pg/mL, preferably 10 pg/mL, preferably 7 pg/mL, and very particularly between 4 and 5 pg/mL, such as for example 4.8 pg/mL.

In humans, the interleukin-10 gene (IL-10) (Gene ID: 3586 updated 27 Mar. 2022) codes for a 178-amino-acid protein (UniProtKB-P22301, amended 23 Feb. 2022) comprising an 18-amino-acid signal peptide. Interleukin 10, sometimes also called CSIF (human cytokine synthesis inhibitory factor) is a protein produced by monocytes and on a lesser scale by lymphocytes and having numerous effects on immunoregulation and inflammation. IL-10 binds to its heterotetrameric receptor comprising IL10RA and IL10RB, leading to phosphorylation of STAT3 via JAK1 and STAT-2. STAT3 is then translocated into the nucleus of the cell to regulate the expression of anti-inflammatory mediators. Advantageously, the reference threshold value of the protein concentration of IL10 is 45 pg/mL, preferably 20 pg/mL, preferably 15 pg/mL, and more preferably between 5 and 10 pg/mL, such as for example 7 pg/mL.

CD14 is a surface antigen essentially expressed by macrophages. It cooperates with other proteins to mediate the innate immune response to bacterial lipopolysaccharide (LPS) and viruses. In humans, CD14 (UniprotKB-P08571, updated 23 Feb. 2022) is encoded by the CD14 gene (Gene ID: 929, updated 27 Mar. 2022). CD14 is a 375-amino-acid protein comprising a signal peptide (the first 19 amino acids of the sequence) and a 30-amino-acid propeptide (sequence between positions 346 to 375). There is a soluble form (amino acids at positions 20 to 367) and the membrane-bound form (amino acids at positions 20 to 345). The soluble form is cleaved by proteases and the cleavage product is a 13 kDa N-terminal fragment called presepsin or sCD14. Presepsin loses its ability to bind to LPS. Presepsin has a different immunogenicity from the soluble form of CD14 and can be distinguished with the aid of specific antibodies (WO2004/044005). Numerous techniques, in particular ELISA methods using specific antibodies recognizing presepsin making it possible to determine the protein concentration of presepsin, are known to those skilled in the art (for review, Memar M Y et al. Biomed Pharmacother, 2019 March; 111:649-656). Advantageously, the reference threshold value of the protein concentration of CD14 is 2000 ng/mL, preferably 1500 ng/mL, preferably 1000 ng/mL, more preferably 800 ng/mL, and very particularly between 700 and 750 ng/mL, such as for example 712 ng/mL.

Interferon gamma-induced protein 10 (IP10), also called C-X-C motif chemokine ligand 10 (CXCL10), or small inducible cytokine B10 (UniProKB-P02778, updated 23 Feb. 2022) is an 8.7 kDa protein which in humans is encoded by the CXCL10 gene (Gene ID: 3627, updated 27 Mar. 2022). IP10 is a pro-inflammatory cytokine that is involved in a wide variety of processes such as chemotaxis, differentiation and activation of peripheral immune cells, regulation of cell growth, apoptosis and modulation of angiostatic effects. It thus plays an important role in viral infections by stimulating the activation and migration of immune cells towards infected sites. Mechanically, CXCL10 binding to the receptor CXCR3 activates G protein-mediated signaling and leads to downstream activation of the phospholipase C-dependent pathway, increased intracellular calcium production, and actin reorganization. Advantageously, the reference threshold value of the protein concentration of IP10 is 200 ng/mL, preferably 150 ng/mL, preferably 100 ng/mL, more preferably 80 ng/mL, and very particularly between 75 and 80 ng/mL, such as for example 78 pg/mL.

Neutrophil gelatinase-associated lipocalin (NGAL/lipocalin-2 Lcn-2) is a 21 kDa human protein from the lipocalin superfamily (UniProtKB-P80188, updated 23 Feb. 2022) comprising a signal peptide consisting of the first 20 amino acids of the sequence. The protein is encoded in humans by the LCN2 gene (Gene ID: 3934, updated 20 Mar. 2022). Lipocalins are transporters of hydrophobic molecules, such as lipids, steroidal hormones, and retinoids. NGAL is a neutrophil gelatinase-associated lipocalin that plays a role in innate immunity by limiting bacterial growth by virtue of sequestration of iron-containing siderophores. Advantageously, the reference threshold value of the protein concentration of NGAL is 300 ng/mL, preferably 200 ng/mL, preferably 150 ng/mL, more preferably 100 ng/mL, and very particularly between 90 and 100 ng/mL, such as for example 96 ng/mL.

Lipopolysaccharide binding protein (LBP) (UniProtKB-P18428, updated 23 Feb. 2022) is encoded in humans by the LBP gene, also known as BPIFD2, (Gene ID: 3929, updated 25 Jan. 2022). LBP is a soluble acute-phase protein that binds to bacterial lipopolysaccharide (or LPS) in order to induce immune responses by presenting LPS to important cell surface pattern recognition receptors called CD14 and TLR42. Advantageously, the reference threshold value of the protein concentration of LBP is 8000 ng/mL, preferably 7000 ng/mL, preferably 6500 ng/mL, more preferably 6200 ng/mL, and very particularly between 6100 and 6200 ng/mL, such as for example 6172 ng/mL.

Calprotectin is a heterocomplex of two calcium-binding proteins, the S100-A8 protein, also known as calgranulin A or MRP8, (UniProtKB-P05109, updated 23 Feb. 2022) encoded in humans by the S100A8 gene (Gene ID: 6279, updated 7 Mar. 2022) and the S100-A9 protein, also known as MRP8/14, (UniProtKB-P06702, updated 23 Feb. 2022) encoded in humans by the S100A9 gene (Gene ID: 6280, updated 27 Mar. 2022). Other names for calprotectin are MRP8-MRP14, calgranulin A and B, cystic fibrosis antigen, L1, 60BB antigen and 27E10 antigen. In the presence of calcium, calprotectin is capable of sequestering the transition metals iron, manganese and zinc by chelation. This sequestration of metals confers antimicrobial properties on the complex. Advantageously, the reference threshold value of the protein concentration of calprotectin is 2000 ng/mL, preferably 1500 ng/mL, preferably 1000 ng/mL, more preferably 800 ng/mL, and very particularly between 740 and 760 ng/mL, such as for example 748 ng/mL.

Gelsolin is a cytosolic (82 kDa) protein (UniProtKB-P06396, updated 23 Feb. 2022) encoded by the GSN gene, also known as ADF or AGEL, (Gene ID: 2394, updated 13 Mar. 2022). Gelsolin, activated by the presence of calcium ions in high concentration, is capable of attaching to actin filaments and creating a local dislocation of these filaments. In addition, it remains attached to the (+) end of the microfilament (polymerization end) and thus avoids rapid repolymerization of actin. Advantageously, the reference threshold value of the protein concentration of gelsolin is 300 μg/mL, preferably 200 μg/mL, more preferably 150 μg/mL, and very particularly between 130 and 140 μg/mL, such as for example 133 μg/mL.

Procalcitonin is a polypeptide consisting of 116 amino acids (12.6 kDa) encoded in humans by the calcitonin gene (Gene ID: 796, updated on 13 Mar. 2022) which is the peptide precursor of calcitonin. It corresponds to the prohormone of calcitonin (CT) (NCBI Sequence Reference: NP_001029124.1, updated 27 Feb. 2022) comprising a 25-amino-acid signal peptide. PCT is primarily synthesized by thyroid C cells and to a lesser extent in neuroendocrine tissue of other organs such as the lungs and intestines. The production of PCT can be stimulated in almost all organs by inflammatory cytokines and more particularly bacterial endotoxins present during sepsis. Advantageously, the reference threshold value of the protein concentration of PCT is 1500 pg/mL, preferably 1000 pg/mL, and more preferably 750 pg/mL, and very particularly between 450 and 550 pg/mL, such as for example 510 pg/mL.

The protein concentration of at least one other protein chosen from: IL-6, IL-10, procalcitonin (PCT), IP10, CD14, LBP, NGAL, calprotectin and gelsolin, preferably NGAL and gelsolin, can be measured by any appropriate method known to those skilled in the art as described above. In particular, the protein concentration is determined by an ELISA method, preferably a microfluidic ELISA method. The antibodies used in the ELISA methods for these biomarkers are known to those skilled in the art and are for example referenced in antibody databases such as the online database Antibodypedia, (Kiermer V. (2008) “Antibodypedia—A web portal to share antibody validation data” Nature methods. 5: 860).

The protein concentration of at least one other protein chosen from: IL-6, IL-10, procalcitonin (PCT), IP10, CD14, LBP, NGAL, calprotectin and gelsolin, preferably NGAL and gelsolin, is compared with a corresponding reference threshold value. Concentrations of the protein PTX3 and of at least one of said other proteins greater than corresponding reference threshold values indicate that the patient has late-onset neonatal sepsis, and preferably that the treatment with the antibiotics is able to be continued. Conversely, concentrations of the protein PTX3 and of at least one of said other proteins lower than corresponding reference threshold values indicate that the patient does not have late-onset neonatal sepsis, and preferably that the treatment with the antibiotics is able to be stopped.

The present disclosure relates to an in vitro method for determining whether a treatment with an antibiotic is able to be stopped in a newborn patient who possibly has late-onset neonatal sepsis and has previously received treatment with the antibiotic, said method comprising determining the protein concentration of PTX-3 in a biological sample previously obtained from said patient and comparing the concentration thus determined with a reference threshold value, characterized in that a protein concentration of PTX-3 lower than said threshold value indicates that the treatment with the antibiotic is able to be stopped and that the patient does not have late-onset neonatal sepsis.

In another aspect, the present application relates to an antibiotic for use thereof in the treatment of a late-onset neonatal sepsis syndrome in a newborn patient, characterized in that said antibiotic is administered to said patient previously evaluated as having late-onset neonatal sepsis by a method as described above.

The present application also relates to a method for treating late-onset neonatal sepsis in a newborn patient, comprising: a) determining the protein concentration of PTX3 and preferably of at least one other protein chosen from: IL-6, IL-10, procalcitonin (PCT), IP10, CD14, LBP, NGAL, calprotectin and gelsolin in a biological sample previously obtained from said patient and comparing the concentration(s) thus determined with one or more reference threshold values, characterized in that a protein concentration of PTX3 and preferably of at least one of said other proteins greater than said reference threshold value(s) indicates that the patient has late-onset neonatal sepsis; b) administering, to said patient evaluated as having late-onset neonatal sepsis, a therapeutically effective amount of an antibiotic.

The present application also relates to the use of an antibiotic in the preparation of a medicament for the treatment of late-onset neonatal sepsis in a newborn patient, characterized in that said antibiotic is administered to said patient after having been evaluated as having late-onset neonatal sepsis by the method described above.

In another particular embodiment, the present application also relates to a method for treating late-onset neonatal sepsis in a newborn patient, comprising:
a) administering to said patient a therapeutically effective amount of an antibiotic,
b) determining the protein concentration of PTX-3 and preferably of at least one other protein chosen from: IL-6, IL-10, procalcitonin (PCT), IP10, CD14, LBP, NGAL, calprotectin and gelsolin in a biological sample obtained from said patient and comparing the concentration(s) thus determined with one or more reference threshold values, characterized in that a protein concentration of PTX3 and preferably of at least one other protein chosen from: IL-6, IL-10, procalcitonin (PCT), IP10, CD14, LBP, NGAL, calprotectin and gelsolin lower than said reference threshold value(s) indicates that the treatment with the antibiotic is able to be stopped in said patient and that said patient does not have late-onset neonatal sepsis,
c) stopping the administration of an antibiotic to said patient for whom the treatment with the antibiotic has been evaluated as being able to be stopped.

Preferably, the antibiotic that may be administered to the newborn patient can include, by way of example, one of the following antibiotics: amoxicillin, amikacin, aztreonam, chloramphenicol, ceftazidime, clindamycin, cefalotin, ciprofloxacin, colistin, cefotetan, cefotaxime, erythromycin, fusidic acid, fosfomycin, cefoxitin, furans, gentamicin, imipenem, kanamycin, lincomycin, cefamandole, minocycline, latamoxef, metronidazole, nalidixic acid, netilmicin, oxacillin, benzylpenicillin, pefloxacin, piperacillin, pristinamycin, rifampicin, spiramycin, sulfamides, streptomycin, trimethoprim, sulfamethoxazole, tetracycline, teicoplanin, ticarcillin, tobramycin, trimethoprim, vancomycin, preferably amoxicillin, gentamicin or cefotaxime.

In the context of the present disclosure, the term “treat” or “treatment”, as used herein, means reversing, relieving, inhibiting the progression or preventing the disorder or state to which this term applies, or reversing, relieving, inhibiting the progression or preventing one or more symptoms of the disorder or state to which this term applies.

As used herein, a “therapeutically effective amount” or an “effective amount” means the amount of a composition which, when administered to a subject to treat a state, disorder or condition, is sufficient to effect treatment. The therapeutically effective amount varies according to the compound, formulation or composition, the disease and its severity, as well as the age, weight, physical condition and responsiveness of the subject to be treated.

The antibiotic described herein can be administered by any means known to persons skilled in the art, including, without limitation, intravenously, orally, intraperitoneally, intramuscularly, parenterally, subcutaneously and topically, preferably intravenously and orally. Thus, the compositions can be formulated in the form of an injectable, topical or ingestible formulation. Administration of the compounds or therapeutic agents to a subject in accordance with the present disclosure may exhibit beneficial effects in a dose-dependent manner. Thus, within broad limits, the administration of larger amounts of compositions should make it possible to obtain greater beneficial biological effects than the administration of a smaller amount. In addition, the efficacy is also envisaged at doses below the level at which toxicity is observed.

It will be appreciated that the specific dosage of the antibiotic in a given case will be adjusted according to the composition administered, the volume of the composition which can be effectively delivered to the administration site, the disease to be treated or inhibited, the state of the subject, and other relevant medical factors that may alter the activity of the compositions or the response of the subject, as is well known to those skilled in art.

In another aspect, the present disclosure relates to a kit comprising a set of reagents making it possible to determine the protein concentration of PTX3 and preferably of at least one of the proteins chosen from: NGAL, IL-10, IL-6, calprotectin, LBP, PCT, sCD14, IP10 or gelsolin, preferably PTX3, NGAL and gelsolin.

Preferably, the reagents comprise antibodies or antibody fragments as defined above for the method.

According to one particular embodiment, the kit comprises a set of reagents making it possible to determine the protein concentration of PTX3 and of at least two other proteins chosen from: NGAL, IL-10, IL-6, calprotectin, LBP, PCT, sCD14, IP10 and gelsolin, preferably from NGAL, IL-10 and gelsolin.

Preferably, the kit comprises a control sample calibrated to contain the threshold value of the concentration of PTX3 as defined above, and optionally one or more other control samples calibrated to each contain the threshold value of the concentration of at least one of the proteins chosen from: NGAL, IL-10, IL-6, calprotectin, LBP, PCT, sCD14, IP10 or gelsolin, preferably NGAL and/or gelsolin and/or IL-10.

More preferably, the kit comprises containers each comprising one or more compounds at a concentration or in an amount that facilitates the reconstitution and/or use of a set of reagents, preferably antibodies, preferentially coupled to a fluorophore or chromogen which allows the specific determination of the protein concentration of PTX3 and preferably of at least one of the proteins chosen from: NGAL, IL-10, IL-6, calprotectin, LBP, PCT, sCD14, IP10 or gelsolin, preferably PTX3, NGAL and gelsolin and the implementation of the method according to the disclosure.

The kit may also comprise instructions indicating the methods for preparing and/or using the reagents for determining the expression level of said genes according to the methods of the disclosure.

The kit according to the present disclosure is therefore particularly suitable in a use for determining whether a treatment with an antibiotic is able to be stopped in a newborn patient who possibly has late-onset neonatal sepsis and has previously received treatment with the antibiotic.

### EXAMPLES

### Materials and Method

### 1. Design of the Study

A prospective, multicenter cohort study was conducted in two neonatal intensive care and resuscitation units (Hôpital Femme Mere Enfant, Hospices Civils de Lyon, Bron; Centre Hospitalier Universitaire de Nantes, Nantes) between 19 Nov. 2017 and 20 Nov. 2020 (clinicaltrials.gov ID: NCT03299751). Newborns older than 7 days who were hospitalized, covered by health insurance and presenting signs suggestive of late-onset neonatal sepsis were included consecutively.

Suspicion of late-onset neonatal sepsis was defined by at least one of the following clinical criteria:
Fever>38° C.
tachycardia>160 bpm,
capillary refill time (CRT)>3 seconds,
gray and/or pale complexion,
bradycardia/apnea syndrome,
abdominal bloating,
rectal bleeding,
hypotonia,
lethargy,
convulsions absent another obvious cause,
increased ventilatory assistance and/or increase in FiO₂,
skin rash or inflammation at the site of puncture of the central venous catheter.

Exclusion criteria in the study included newborns treated with antibiotics for a bacteriologically documented infection at the time of or within 48 hours prior to blood collection, newborns who had undergone surgical intervention within the preceding 7 days, newborns vaccinated within the preceding 7 days, newborns who had received systemic corticosteroid therapy within 48 hours prior to collection, and newborns exhibiting severe combined immunodeficiency.

At inclusion, investigators recorded demographic data, medical history, disease history, neonatal data, the presence of neonatal transfusion within the 7 days prior to inclusion, and physical examination data.

The results of additional tests performed (chest X-ray, bacteriological samples, C-reactive protein (CRP), white blood cell count, absolute neutrophil count) were also recorded. Standard care, according to the assessment of the investigators, included blood culture and in some patients the performance of other tests (CRP, urine analysis, lumbar puncture, stool culture, and chest x-ray, for example). The decision of whether or not to treat the newborn with antibiotics was left to the discretion of the physician. At 48 hours post-inclusion, clinical re-evaluation was performed, including clinical data and also intubation data, the biological CRP and bacteriology assay, and antibiotic therapy data.

### 2. Adjudication Procedure

In the absence of a gold standard for the diagnosis of infection in newborns, a reference standard was established by a panel of experts, following existing medical recommendations.

Thus, an independent adjudication committee was formed with three pediatric neonatal resuscitation experts, having expertise in neonatal infectious diseases and being independent from the team carrying out the inclusion. The experts assigned each included newborn one of the following 3 diagnostic categories: (i) confirmed infection, (ii) non-confirmed infection, (iii) infection undetermined. The classification by the experts of the adjudication committee was based on clinical data collected at inclusion and at 48 h blinded from the results of biomarker measurement and the decision of their peers. The final diagnosis was determined by the majority agreement of the committee (at least two classifications out of three in agreement). In the event of a non-majority, the three experts reached a consensus decision on the diagnosis.

### 3. Sample Collection and Biomarker Measurements

At inclusion, 0.4 mL of blood was collected at the time of venipuncture prescribed within the context of standard care, for assaying the following 11 protein markers:
Interleukin 10 (IL-10),
Procalcitonin (PCT),
Interleukin 6 (IL-6),
Interferon gamma induced protein 10 (IP-10) (also referred to as CXCL10),
Neutrophil gelatinase associated lipocalin (NGAL) (also referred to as lipocalin-2),
Pentraxin 3 (PTX3) (also referred to as TSG-14),
Presepsin (sCD14),
Lipopolysaccharide binding protein (LBP),
Calprotectin,
Gelsolin, and
Interleukin 27 (IL-27).

The assaying of these 11 biomarkers was carried out in a central laboratory (HCL-bioMerieux Joint Research Unit, Lyon, France), in serum samples prepared from 0.4 mL of whole blood collected in BD Microtainer™ serum separator tubes (Becton Dickinson, reference BD365968). After 2 hours of coagulation at ambient temperature and centrifugation at 2500 g for 10 min, the sera were aliquoted and stored at −80° C. until biomarker measurements.

Concentrations of IL-10, PCT, IP-10, IL-6, NGAL, PTX3, presepsin and LBP were measured in the serum samples via the Simple Plex™ (Protein simple©, CA, USA) automated immunoassay platform in accordance with the manufacturer's instructions. Simple Plex™ is an integrated immunoassay system taking the form of a disposable microfluidic cartridge and an automated analyzer, the ELLA instrument.

Quantification of IL10, PCT, IP-10 and IL-6 was performed simultaneously in a multiplex cartridge format using 50 μl of twice-diluted serum. The concentrations of NGAL, PTX3, presepsin, and LBP were measured in a multiplex cartridge format using 50 μL of serum diluted to 1:400.

Gelsolin concentrations were measured using the human GS (Gelsolin) ELISA kit (Elabsciences®) using serum diluted to 1:2000, in accordance with the manufacturer's instructions.

Calprotectin concentrations were measured using the Human S100A8/S100A9 Heterodimer Quantikine ELISA kit (R&D Systems, MN, USA) with a serum diluted to 1:200.

Lastly, the levels of IL-27 were measured using the DuoSet ELISA kit (R&D Systems, MN, USA) with a twice-diluted serum. All measurements were performed in duplicate by manual ELISA.

### 4. Statistical Analyses

The data are described by median and range (quantitative data) and numbers and percentages for categorical data. The comparison of the marker values between the “confirmed infection” and “confirmed absence of infection” groups is carried out for exploratory purposes by a Shapiro-Wilk test.

Evaluation of the diagnostic capacity of clinical signs/markers is performed on groups of patients with “confirmed infection” or “non-confirmed infection”.

Univariate logistic regressions were performed to evaluate the association between symptoms and confirmed infection; the association was quantified by an odds ratio with a 95% confidence interval thereof. Symptoms which have a p-value of less than 0.2 in univariate analysis and little collinearity and which are clinically relevant were combined through a multivariate logistic regression model.

Markers were combined together in order to predict confirmed infection by a logistic regression model assuming an additive effect on the linear predictor scale. Transformations of the markers (log) were considered in order to satisfy the hypotheses of the model. Predictions from logistic regression models are then used as a new marker summarizing the combination of the markers. All combinations of 2, 3 or 4 markers were considered.

Marker performance, marker combinations, symptoms and symptom combinations were evaluated by constructing ROC curves, and calculating areas under the ROC curve and partial areas under the ROC curve (area in the zone where sensitivity is greater than 0.90) with the associated confidence intervals. For each marker and marker combination, the threshold associated with the highest specificity and a sensitivity of at least 0.9 was determined. The following was evaluated at this threshold: specificity, positive predictive value (PPV), negative predictive value (NPV), and positive and negative likelihood ratios with their associated 95% confidence intervals.

The combination of markers is carried out within the context of the study on a training set, which will require confirmation on a subsequent confirmation set.

Statistical analyses were carried out with R and SAS software.

### 5. Ethics

Written informed consent was obtained from at least one of the patients' parents or legal guardians. The study was approved by the Ethics Committee (Comité de Protection des Personnes [CPP]) Sud-Ouest et Outremer III under registration number 2017-A02492-51, and was conducted in accordance with the recommendations for good clinical practice and the Declaration of Helsinki. The study has been registered on clinicaltrials.gouv.fr under the number NCT03299751.

### Results

### 1. Presentation of the Cohort of Patients and of the Classification Carried Out by the Adjudication Committee

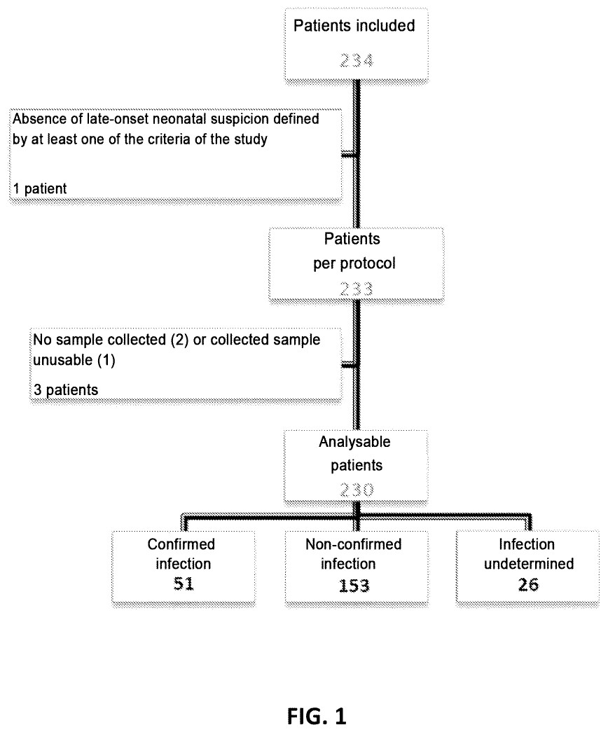

As shown in [FIG. 1], out of 234 patients included, 230 were able to be classified by the adjudication committee into the three diagnostic categories defined above. In particular, 51 patients were classified as having a confirmed infection, 153 patients as having a non-confirmed infection (i.e., confirmed absence of infection), and 26 patients for whom it was not possible to conclude whether an infection was present or absent (i.e., undetermined infection).

### 2. Demographic and Clinical Characteristics of the Patients on Admission

The overall demographic and clinical characteristics of the patients on admission are presented in [Table 1] below:

TABLE 1

| | | | | | Confirmed | | | |
|---|---|---|---|---|---|---|---|---|
| | All patients | | Confirmed | | Absence of | | Infection | |
| | (N = 230) | | Infection | | Infection | | Undetermined | |
| Calculated age (days) N | | | 51 | | 153 | | 26 | |
| Calculated age (days) median (range) | 14.0 | (7.0-178.0) | 11 | (7-159) | 15 | (7-178) | 14 | (7-69) |
| Sex | | | 51 | | 153 | | 26 | |
| Male | 137 | (59.6%) | 39 | (76.5%) | 86 | (56.2%) | 12 | (46.2%) |
| Female | 93 | (40.4) | 12 | (23.5%) | 67 | (43.8%) | 14 | (53.8%) |
| Gestational Age (GA) N | | | 51 | | 153 | | 26 | |
| Gestational Age (GA) median (range) | 27.0 | (23.0-41.0) | 27 | (24-41) | 28 | (23-41) | 26.5 | (24-38) |
| Birth weight (g) N | | | 51 | | 153 | | 26 | |
| Birth weight (g) median (range) | 940.0 | (450.0-4660.0) | 960 | (530-3400) | 930 | (450-4660) | 902.5 | (490-3430) |
| Birth weight (2 classes) | | | 51 | | 153 | | 26 | |
| <1500 g | 184 | (80.0%) | 39 | (76.5%) | 126 | (82.4%) | 19 | (73.1%) |
| >=1500 g | 46 | (20.0) | 12 | (23.5%) | 27 | (17.6%) | 7 | (26.9%) |
| Apgar 5 minutes N | | | 51 | | 151 | | 25 | |
| Apgar 5 minutes (n = 227) median (range) | 8.0 | (1.0-10.0) | 9 | (1-10) | 8 | (1-10) | 8 | (4-10) |
| Intrauterine growth restriction | | | 51 | | 153 | | 26 | |
| yes | 67 | (29.1%) | 10 | (19.6%) | 52 | (34.0%) | 5 | (19.2%) |
| Histological chorioamnionitis (n = 218) | | | 48 | | 146 | | 24 | |
| yes | 36 | (16.5) | 7 | (14.6%) | 26 | (17.8%) | 3 | (12.5%) |
| Congenital malformation | | | 51 | | 153 | | 26 | |
| yes | 41 | (17.8) | 13 | (25.5%) | 25 | (16.3%) | 3 | (11.5%) |
| Surgery prior to inclusion | | | 51 | | 153 | | 26 | |
| yes | 35 | (15.2) | 17 | (33.3%) | 15 | (9.8%) | 3 | (11.5%) |
| Time between surgery and inclusion | | | 17 | | 15 | | 3 | |
| (days) N | | | | | | | | |
| Time between surgery and inclusion | 15.0 | (4.0-63.0) | 16 | (6-63) | 15 | (6-43) | 6 | (4-52) |
| (days) (n = 35) median (range) | | | | | | | | |
| Neonatal transfusion within the 7 days | | | 51 | | 153 | | 26 | |
| preceding the inclusion | | | | | | | | |
| yes | 40 | (17.4) | 11 | (21.6%) | 23 | (15.0%) | 6 | (23.1%) |

### 3. Clinical State of the Patients at the Time of Sample Collection

The clinical state of the patients at the time of sample collection is presented in [Table 2] below:

TABLE 2

| | | | Confirmed | |
|---|---|---|---|---|
| | All | Confirmed | Absence of | Infection |
| | patients | Infection | Infection | Undetermined |
| Presence of a central | | 50 | 153 | 26 |
| venous catheter (n = 229) | | | | |
| | 146 | 44 (88.0%) | 86 | 16 (61.5%) |
| | (63.8) | | (56.2%) | |
| Fever >=38° C. (n = 229) | | 50 | 153 | 26 |
| | 84 | 25 (50.0%) | 50 | 9 (34.6%) |
| | (36.7) | | (32.7%) | |
| Tachycardia >160/min | | 51 | 153 | 26 |
| | 124 | 33 (64.7%) | 74 | 17 (65.4%) |
| | (53.9) | | (48.4%) | |
| CRT >3 seconds (n = 226) | | 51 | 150 | 25 |
| | 18 | 10 (19.6%) | 5 | 3 (12.0%) |
| | (8.0) | | (3.3%) | |
| Gray/pale complexion (n = 227) | | 51 | 152 | 24 |
| | 56 | 18 (35.3%) | 29 | 9 (37.5%) |
| | (24.7) | | (19.1%) | |
| Bradycardia/apnea syndrome (n = 229) | | 51 | 153 | 25 |
| | 111 | 23 (45.1%) | 78 | 10 (40.0%) |
| | (48.5) | | (51.0%) | |
| Increase in ventilatory assistance | | 51 | 153 | 26 |
| and/or in FiO2 | | | | |
| | 107 | 26 (51.0%) | 63 | 18 (69.2%) |
| | (46.5) | | (41.2%) | |
| Digestive disorders (bloating, | | 51 | 153 | 26 |
| vomiting or rectorrhagia) | | | | |
| | 120 | 26 (51.0%) | 81 | 13 (50.0%) |
| | (52.2) | | (52.9%) | |
| Hypotonia or Lethargy (n = 229) | | 51 | 153 | 25 |
| | 38 | 14 (27.5%) | 17 | 7 (28.0%) |
| | (16.6) | | (11.1%) | |
| Skin rash | | 51 | 153 | 26 |
| | 5 | 1 (2.0%) | 3 | 1 (3.8%) |
| | (2.2) | | (2.0%) | |

### 4. Additional Examinations

The results of the additional biological analyses are presented in [Table 3] below:

TABLE 3

| | | | Confirmed | |
|---|---|---|---|---|
| | All | Confirmed | Absence of | Infection |
| | patients | Infection | Infection | Undetermined |
| CRP (mg/L) N | | 41 | 123 | 23 |
| CRP (mg/L) (n = 187) median | 1.0 | 14.6 | 1 | 5.6 |
| (range) | (0.0-207.0) | (0-207) | (0-22.9) | (0-165.9) |
| Leukocyte count | | 31 | 88 | 14 |
| (10/L) N | | | | |
| Leukocyte count | 13.5 | 14.48 | 12.65 | 15.98 |
| (10/L) (n = 133) median (range) | (2.4-40.1) | (2.42-40.12) | (2.94-30.76) | (5.67-33.3) |
| Polynuclear neutrophils | | 21 | 75 | 10 |
| (PNN)(10/L) N | | | | |
| PNN (10/L) (n = 106) median | 5.5 | 7.56 | 4.62 | 4 |
| (range) | (0.9-22.1) | (1.18-22.07) | (0.93-15.44) | (1.01-21.98) |
| Lymphocytes (10/L) N | | 21 | 75 | 10 |
| Lymphocytes (10/L) (n = 106) | 5.1 | 4.02 | 5.44 | 3.66 |
| median (range) | (0.8-14.9) | (0.79-6.73) | (1.14-14.9) | (1.17-7.93) |

### 5. Results of the Blood Cultures and of the Antibiotic Therapy

The results are presented in [Table 4] below:

TABLE 4

| | | | | | Confirmed | | | |
|---|---|---|---|---|---|---|---|---|
| | | | Confirmed | | absence of | | Infection | |
| | All patients | | infection | | infection | | undetermined | |
| Blood culture n/N (%) | | | | | | | | |
| Not performed | 2/230 | (0.9%) | 0/51 | (0%) | 2/153 | (1.3%) | 0/26 | (0%) |
| Sterile | 180/230 | (78%) | 8/51 | (15.7%) | 148/153 | (96.7%) | 24/26 | (92.3%) |
| Positive | 48/230 | (20.9%) | 43/51 | (84.3%) | 3/153 | (2%) | 2/26 | (7.7%) |
| Staphylococcus aureus | 8/228 | (3.5%) | 8/51 | (15.7%) | 0/151 | (0%) | 0/26 | (0%) |
| (n = 228) | | | | | | | | |
| Coagulase-negative | 35/228 | (15.4%) | 30/51 | (58.8%) | 3/151 | (2.0%) | 2/26 | (7.7%) |
| staphylococci (n = 228) | | | | | | | | |
| Gram-negative Bacilli | 3/228 | (1.3%) | 3/51 | (5.9%) | 0/151 | (0%) | 0/26 | (0%) |
| (n = 228) | | | | | | | | |
| Other Gram-positive | 2/228 | (0.9%) | 2/51 | (3.9%) | 0/151 | (0%) | 0/26 | (0%) |
| microorganism (n = 228) | | | | | | | | |
| Candida albicans | 1/228 | (0.4%) | 1/51 | (2.0%) | 0/151 | (0%) | 0/26 | (0%) |
| (n = 228) | | | | | | | | |
| Antibiotic treatment at | 230 | | 51 | | 153 | | 26 | |
| 48 h n/N (%) | | | | | | | | |
| No | 117 | (50.9%) | 0 | (0%) | 111 | (72.5%) | 6 | (23.1%) |
| Yes | 113 | (49.1%) | 51 | (100%) | 42 | (27.5%) | 20 | (76.9%) |
| Vancomycin | 98 | (42.6%) | 48 | (94.1%) | 36 | (23.5%) | 14 | (53.8%) |
| Amikacin | 80 | (34.8%) | 35 | (68.6%) | 32 | (20.9%) | 13 | (50.0%) |
| Cefotaxime | 41 | (17.8%) | 20 | (39.2%) | 13 | (8.5%) | 8 | (30.8%) |
| other beta-lactams | 18 | (7.8%) | 8 | (15.7%) | 5 | (3.3%) | 5 | (19.2%) |
| Metronidazole | 2 | (0.9%) | 1 | (2.0%) | 1 | (0.7%) | 0 | (0%) |
| Others | 20 | (8.7%) | 9 | (17.6%) | 6 | (3.9%) | 5 | (19.2%) |

Thus, 84% of the patients classified as having a confirmed infection by the adjudication committee have a positive blood culture. This percentage does not reach 100% due to the limitations of blood culture, in particular the false negative rate and also the volume of blood necessary for an optimal culture which cannot always be taken from very-low-weight newborns with insufficient total blood volume.

The inventors also note that all of the patients classified as having a confirmed infection had indeed received antibiotic treatment, confirming that it is not necessary to have available new biomarkers for the positive diagnosis of the infection. However, among the patients classified as having a confirmed absence of infection, 27.5% had received antibiotic therapy when it was not necessary, thus confirming the need to have available new biomarkers making it possible to stop antibiotic treatment at an early stage.

This is because, when there is a suspicion of late-onset neonatal sepsis, the medical recommendations consist in treating the newborn with antibiotics until the results of the blood culture are available, which in practice represents a delay of 48 h.

Consequently, in the case where the blood culture turns out to be sterile and the decision is taken by the physician to stop the antibiotic treatment, the newborn has still received the treatment for about 48 h.

In view of the harmful consequences of the use of antibiotics on newborns, namely in particular mortality during hospitalization (see in particular Ting et al. JAMA Pediatr. 2016 Dec. 1; 170(12):1181-1187—doi: 10.1001/jamapediatrics.2016.2132) or the alteration of the microbiota with long-term effects (see in particular Kummeling et al. https://doi.org/10.1542/peds.2006-0896), there is a need to be able to stop treatment as early as possible from the time that it was prescribed.

As demonstrated in the section below, this need is all the more important as clinical signs alone do not exhibit sufficient performance to allow a decision to be taken on whether to stop the antibiotic treatment.

### 6. Performance of the Model Based on Clinical Signs Only

The clinical model is derived from a multivariate analysis based on the clinical signs having a p-value of less than 0.2 in univariate analysis. Thus, the clinical signs that have been retained are presented in [Table 5] below:

TABLE 5

| | | lower | upper | |
|---|---|---|---|---|
| | OR | 95% CI | 95% CI | P-value |
| Tachycardia Yes | 1.579 | 0.791 | 3.208 | 0.196 |
| CRT >3 sec Yes | 4.016 | 1.151 | 15.167 | 0.029 |
| Hypotonia/lethargy Yes | 2.100 | 0.806 | 5.213 | 0.125 |

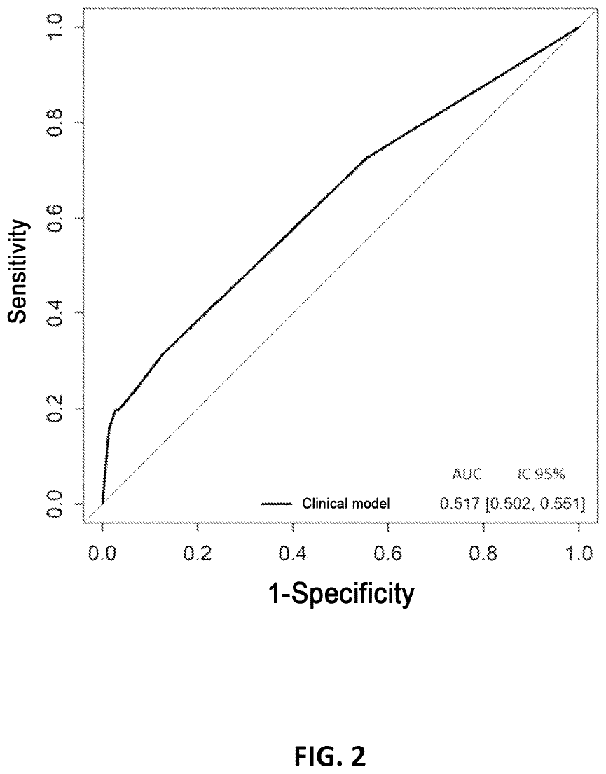

The ROC curve of the clinical signs is presented in [FIG. 2] and confirms that the clinical signs alone do not have sufficient performance for determining the stopping of the antibiotic treatment in the event of suspicion of late-onset neonatal sepsis.

### 7. Analysis of the Performance of the Biomarkers

### 7.1. Description of the Biomarkers According to Patient Status

### [Table 6]

TABLE 6

| | | | | pValue |
|---|---|---|---|---|
| | | | | (Confirmed |
| | | | | Infection |
| | | | Confirmed | vs Confirmed |
| | | Confirmed | Absence of | Absence of |
| | All patients | infection | Infection | Infection) |
| PTX3 (pg/mL) N | | 49 | 153 | |
| PTX3 (pg/mL) (n = | 2416.0 | 3951 | 1856 | <.001 |
| 228) median (range) | (427.0-150366) | (965-150366) | (427-14196) | |
| NGAL (ng/mL) N | | 49 | 153 | |
| NGAL (ng/mL) (n = | 101.7 | 203.5 | 79.7 | <.001 |
| 228) median (range) | (30.1-1342.6) | (44.8-1342.6) | (30.1-451.7) | |
| IL10 (pg/mL) N | | 51 | 153 | |
| IL10 (pg/mL) median | 8.4 | 53.2 | 7.13 | <.001 |
| (range) | (2.5-3861.0) | (4.4-3861) | (2.48-158) | |
| IL6 (pg/mL) N | | 51 | 153 | |
| IL6 (pg/mL) median | 9.2 | 105 | 5.52 | <.001 |
| (range) | (0.3-63292.0) | (1.61-63292) | (0.3-963) | |
| PCT (pg/mL) N | | 51 | 153 | |
| PCT (pg/mL) median | 1524.0 | 2392 | 1237 | <.001 |
| (range) | (137.0-24472.0) | (226-24472) | (137-8396) | |
| IP10 (pg/mL) N | | 51 | 153 | |
| IP10 (pg/mL) median | 114.5 | 218 | 103 | <.001 |
| (range) | (17.7-5876.0) | (29.2-5876) | (17.7-1299) | |
| sCD14 (ng/mL) N | | 49 | 153 | |
| sCD14 (ng/mL) (n = | 887.0 | 1163.2 | 800.3 | <.001 |
| 228) median (range) | (372.5-3617.3) | (491.2-3617.3) | (372.5-2222.2) | |
| LBP (ng/mL) N | | 49 | 153 | |
| LBP (ng/mL) (n = | 9369.0 | 18763 | 7252 | <.001 |
| 228) median (range) | (2142.0-70715.0) | (2866-79715) | (2142-64445) | |
| Calprotectin | | 51 | 150 | |
| (ng/mL) N | | | | |
| Calprotectin | 1587.5 | 2702.4 | 1302.2 | <.001 |
| (ng/mL) (n = 227) | (130.3-29815.0) | (477.6-22845) | (150.5-29815) | |
| medan (range) | | | | |
| Gelsolin (μg/mL) N | | 49 | 150 | |
| Gelsolin (μg/mL) | 207.3 | 209.3 | 203.45 | NS |
| (n = 225) median | (69.9-1595.9) | (69.9-1106.3) | (90.5-1595.9) | |
| (range) | | | | |
| IL27 (pg/mL) N | | 27 | 95 | |
| IL27 (pg/mL) (n = | 312.2 | 485.1 | 266.4 | NS |
| 136) median (range) | (0.0-101063) | (107.2-20996.1) | (0-11228.6) | |

### 7.2. ROC Curves and AUC

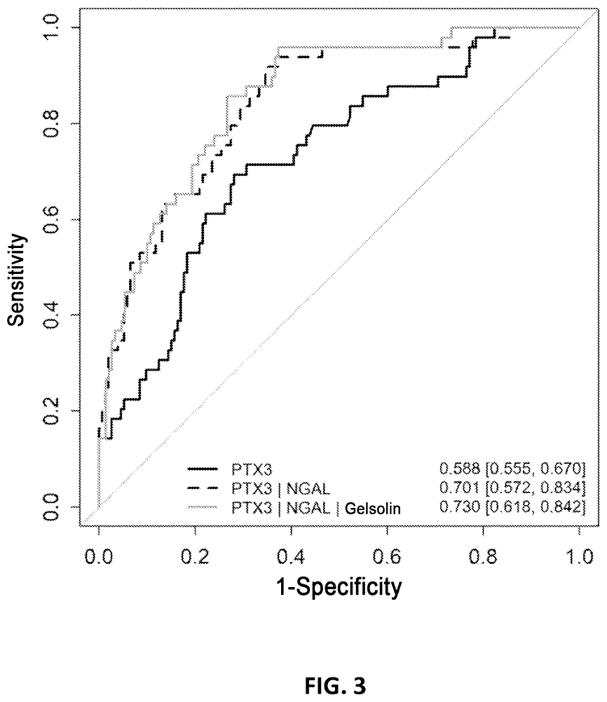

The analyses are made considering the outcome “occurrence of a confirmed infection” (versus confirmed absence of infection). The performances of the biomarkers taken one by one were evaluated in particular by the area under the ROC curve and are presented in part in FIG. 3 and repeated in table 7 below.

For each marker, the threshold retained is that exhibiting the highest specificity and leading to a sensitivity of at least 0.895. The sensitivity, specificity, positive and negative predictive values and also positive and negative likelihood ratios were estimated at the optimal threshold, with their associated 95% confidence intervals.

### Lone Biomarkers

TABLE 7

| Biomarker | AUC | Sensitivity | Specificity | CutOff |
|---|---|---|---|---|
| PTX3 (pg/mL) | 0.732 [0.652; 0.811] | 0.898 | 0.294 [0.223; 0.373] | 1266 |
| NGAL (ng/mL) | 0.829 [0.760; 0.898] | 0.898 | 0.614 [0.532; 0.692] | 95.9 |
| IL10 (pg/mL) | 0.845 [0.777; 0.914] | 0.902 | 0.536 [0.454; 0.617] | 7.27 |
| IL6 (pg/mL) | 0.864 [0.799; 0.929] | 0.902 | 0.438 [0.358; 0.520] | 4.74 |
| PCT (pg/mL) | 0.666 [0.568; 0.763] | 0.902 | 0.137 [0.087; 0.202] | 510 |
| IP10 (pg/mL) | 0.732 [0.644; 0.821] | 0.902 | 0.268 [0.200; 0.345] | 77.7 |
| sCD14 (ng/mL) | 0.734 [0.647; 0.821] | 0.898 | 0.340 [0.265; 0.421] | 712.2 |
| LBP (ng/mL) | 0.786 [0.707; 0.865] | 0.898 | 0.346 [0.271; 0.427] | 6172 |
| Calprotectin | 0.690 [0.609; 0.772] | 0.902 | 0.287 [0.216; 0.366] | 748.1 |
| (ng/mL) | | | | |
| Gelsolin | 0.537 [0.440; 0.634] | 0.898 | 0.073 [0.037; 0.127] | 133.3 |
| (μg/mL) | | | | |

With regard to the performances, and in particular by means of the identified threshold, the PTX3 biomarker according to the invention can thus be used as a marker of late-onset neonatal sepsis in newborns, thus complementing the tools available to practitioners for identifying infections in a neonatal population.

### Combinations of Biomarkers

The biomarker PTX3 was combined with the other biomarkers within the context of a logistic regression model, assuming an additive effect of the markers after logarithmic transformation, and the performance results are presented in [Table 8] below:

TABLE 8

| | N | | Partial | | | | | | |
|---|---|---|---|---|---|---|---|---|---|
| | obs | AUC | AUC | Sens. | Spec. | NPV | PPV | LR+ | LR− |
| Models (2 | | | | | | | | | |
| biomarkers) | | | | | | | | | |
| PTX3 | 202 | 0.732 | 0.588 | 0.898 | 0.294 | 0.900 | 0.289 | 1.272 | 0.347 |
| | | [0.652; | [0.555; | | | | | [1.107; | [0.146; |
| | | 0.811] | 0.670] | | | | | 1.462] | 0.825] |
| PTX3_NGAL | 202 | 0.845 | 0.701 | 0.898 | 0.654 | 0.952 | 0.454 | 2.592 | 0.156 |
| | | [0.783; | [0.572; | | | | | [2.045; | [0.067; |
| | | 0.906] | 0.834] | | | | | 3.286] | 0.361] |
| PTX3_IL10 | 202 | 0.864 | 0.682 | 0.898 | 0.510 | 0.940 | 0.370 | 1.832 | 0.200 |
| | | [0.801; | [0.602; | | | | | [1.519; | [0.086; |
| | | 0.928] | 0.764] | | | | | 2.209] | 0.466] |
| PTX3_IL6 | 202 | 0.870 | 0.670 | 0.898 | 0.562 | 0.945 | 0.396 | 2.051 | 0.182 |
| | | [0.808; | [0.602; | | | | | [1.674; | [0.078; |
| | | 0.932] | 0.774] | | | | | 2.512] | 0.421] |
| PTX3_Calprotectin | 199 | 0.761 | 0.635 | 0.898 | 0.460 | 0.932 | 0.352 | 1.663 | 0.222 |
| | | [0.688; | [0.559; | | | | | [1.396; | [0.095; |
| | | 0.834] | 0.728] | | | | | 1.981] | 0.518] |
| PTX3_LBP | 202 | 0.812 | 0.630 | 0.898 | 0.510 | 0.940 | 0.370 | 1.832 | 0.200 |
| | | [0.742; | [0.554; | | | | | [1.519; | [0.086; |
| | | 0.883] | 0.749] | | | | | 2.209] | 0.466] |
| PTX3_PCT | 202 | 0.751 | 0.627 | 0.898 | 0.484 | 0.937 | 0.358 | 1.739 | 0.211 |
| | | [0.676; | [0.558; | | | | | [1.452; | [0.090; |
| | | 0.827] | 0.727] | | | | | 2.082] | 0.492] |
| PTX3_sCD14 | 202 | 0.785 | 0.616 | 0.898 | 0.444 | 0.932 | 0.341 | 1.616 | 0.230 |
| | | [0.710; | [0.546; | | | | | [1.363; | [0.098; |
| | | 0.859] | 0.720] | | | | | 1.916] | 0.537] |
| PTX3_IP10 | 202 | 0.787 | 0.604 | 0.898 | 0.405 | 0.925 | 0.326 | 1.510 | 0.252 |
| | | [0.710; | [0.537; | | | | | [1.285; | [0.107; |
| | | 0.864] | 0.699] | | | | | 1.774] | 0.591] |
| PTX3_Gelsolin | 199 | 0.732 | 0.596 | 0.898 | 0.287 | 0.896 | 0.291 | 1.259 | 0.356 |
| | | [0.652; | [0.562; | | | | | [1.096; | [0.149; |
| | | 0.812] | 0.669] | | | | | 1.446] | 0.848] |
| Models (3 | | | | | | | | | |
| biomarkers) | | | | | | | | | |
| PTX3 | 202 | 0.732 | 0.588 | 0.898 | 0.294 | 0.900 | 0.289 | 1.272 | 0.347 |
| | | [0.652; | [0.555; | | | | | [1.107; | [0.146; |
| | | 0.811] | 0.670] | | | | | 1.462] | 0.825] |
| PTX3_NGAL_{—} | 199 | 0.856 | 0.730 | 0.898 | 0.640 | 0.950 | 0.449 | 2.494 | 0.159 |
| Gelsolin | | [0.800; | [0.618; | | | | | [1.975; | [0.069; |
| | | 0.913] | 0.842] | | | | | 3.150] | 0.369] |
| PTX3_NGAL_{—} | 199 | 0.849 | 0.718 | 0.898 | 0.673 | 0.953 | 0.473 | 2.749 | 0.152 |
| Calprotectin | | [0.790; | [0.588; | | | | | [2.144; | [0.066; |
| | | 0.909] | 0.840] | | | | | 3.524] | 0.350] |
| PTX3_NGAL_PCT | 202 | 0.846 | 0.708 | 0.898 | 0.647 | 0.952 | 0.449 | 2.544 | 0.158 |
| | | [0.786; | [0.582; | | | | | [2.013; | [0.068; |
| | | 0.907] | 0.833] | | | | | 3.216] | 0.365] |
| PTX3_IL10_NGAL | 202 | 0.885 | 0.704 | 0.898 | 0.647 | 0.952 | 0.449 | 2.544 | 0.158 |
| | | [0.829; | [0.604; | | | | | [2.013; | [0.068; |
| | | 0.942] | 0.825] | | | | | 3.216] | 0.365] |
| PTX3_IL10_PCT | 202 | 0.879 | 0.701 | 0.898 | 0.608 | 0.949 | 0.423 | 2.290 | 0.168 |
| | | [0.822; | [0.638; | | | | | [1.840; | [0.072; |
| | | 0.936] | 0.796] | | | | | 2.850] | 0.389] |
| PTX3_IL10_{—} | 199 | 0.870 | 0.701 | 0.898 | 0.533 | 0.941 | 0.386 | 1.924 | 0.191 |
| Gelsolin | | [0.808; | [0.603; | | | | | [1.583; | [0.082; |
| | | 0.931] | 0.787] | | | | | 2.339] | 0.445] |
| PTX3_IL6_PCT | 202 | 0.882 | 0.694 | 0.898 | 0.608 | 0.949 | 0.423 | 2.290 | 0.168 |
| | | [0.824; | [0.628; | | | | | [1.840; | [0.072; |
| | | 0.939] | 0.795] | | | | | 2.850] | 0.389] |
| PTX3_NGAL_LBP | 202 | 0.850 | 0.690 | 0.898 | 0.641 | 0.951 | 0.444 | 2.498 | 0.159 |
| | | [0.789; | [0.587; | | | | | [1.982; | [0.069; |
| | | 0.911] | 0.822] | | | | | 3.149] | 0.369] |
| PTX3_IL10_LBP | 202 | 0.878 | 0.687 | 0.898 | 0.562 | 0.945 | 0.396 | 2.051 | 0.182 |
| | | [0.819; | [0.627; | | | | | [1.674; | [0.078; |
| | | 0.938] | 0.783] | | | | | 2.512] | 0.421] |
| PTX3_IL10_IP10 | 202 | 0.864 | 0.685 | 0.898 | 0.503 | 0.939 | 0.367 | 1.808 | 0.203 |
| | | [0.800; | [0.605; | | | | | [1.502; | [0.087; |
| | | 0.928] | 0.762] | | | | | 2.176] | 0.472] |
| PTX3_NGAL_sCD14 | 202 | 0.845 | 0.683 | 0.898 | 0.634 | 0.951 | 0.440 | 2.453 | 0.161 |
| | | [0.783; | [0.563; | | | | | [1.951; | [0.070; |
| | | 0.908] | 0.821] | | | | | 3.084] | 0.373] |
| PTX3_IL10_CD14 | 202 | 0.866 | 0.681 | 0.898 | 0.536 | 0.943 | 0.383 | 1.935 | 0.190 |
| | | [0.804; | [0.601; | | | | | [1.593; | [0.082; |
| | | 0.929] | 0.771] | | | | | 2.351] | 0.443] |
| PTX3_NGAL_IP10 | 202 | 0.856 | 0.679 | 0.898 | 0.693 | 0.955 | 0.484 | 2.923 | 0.147 |
| | | [0.793; | [0.537; | | | | | [2.263; | [0.064; |
| | | 0.918] | 0.838] | | | | | 3.776] | 0.340] |
| PTX3_NGAL_IL6 | 202 | 0.880 | 0.679 | 0.898 | 0.634 | 0.951 | 0.440 | 2.453 | 0.161 |
| | | [0.821; | [0.604; | | | | | [1.951; | [0.070; |
| | | 0.939] | 0.816] | | | | | 3.084] | 0.373] |
| PTX3_IL6_{—} | 199 | 0.866 | 0.673 | 0.898 | 0.493 | 0.937 | 0.367 | 1.772 | 0.207 |
| Calprotectin | | [0.804; | [0.602; | | | | | [1.474; | [0.089; |
| | | 0.929] | 0.771] | | | | | 2.130] | 0.482] |
| PTX3_IL10_{—} | 199 | 0.864 | 0.673 | 0.898 | 0.467 | 0.933 | 0.355 | 1.684 | 0.219 |
| Calprotectin | | [0.800; | [0.625; | | | | | [1.411; | [0.094; |
| | | 0.927] | 0.758] | | | | | 2.010] | 0.511] |
| PTX3_IL6_{—} | 199 | 0.871 | 0.671 | 0.898 | 0.593 | 0.947 | 0.419 | 2.208 | 0.172 |
| Gelsolin | | [0.810; | [0.609; | | | | | [1.781; | [0.074; |
| | | 0.932] | 0.780] | | | | | 2.738] | 0.399] |
| PTX3_IL6_IL10 | 202 | 0.883 | 0.670 | 0.898 | 0.588 | 0.947 | 0.411 | 2.181 | 0.173 |
| | | [0.823; | [0.596; | | | | | [1.765; | [0.075; |
| | | 0.942] | 0.801] | | | | | 2.695] | 0.402] |
| PTX3_PCT_IP10 | 202 | 0.809 | 0.664 | 0.898 | 0.516 | 0.940 | 0.373 | 1.857 | 0.198 |
| | | [0.741; | [0.583; | | | | | [1.537; | [0.085; |
| | | 0.876] | 0.770] | | | | | 2.243] | 0.460] |
| PTX3_IL6_CD14 | 202 | 0.869 | 0.663 | 0.898 | 0.556 | 0.944 | 0.393 | 2.020 | 0.184 |
| | | [0.807; | [0.593; | | | | | [1.653; | [0.079; |
| | | 0.931] | 0.774] | | | | | 2.470] | 0.427] |
| PTX3_PCT_{—} | 199 | 0.773 | 0.659 | 0.898 | 0.467 | 0.933 | 0.355 | 1.684 | 0.219 |
| Calprotectin | | [0.703; | [0.599; | | | | | [1.411; | [0.094; |
| | | 0.843] | 0.742] | | | | | 2.010] | 0.511] |
| PTX3_IL6_IP10 | 202 | 0.869 | 0.658 | 0.898 | 0.549 | 0.944 | 0.389 | 1.991 | 0.186 |
| | | [0.806; | [0.589; | | | | | [1.632; | [0.080; |
| | | 0.932] | 0.774] | | | | | 2.429] | 0.432] |
| PTX3_IL6_LBP | 202 | 0.870 | 0.656 | 0.898 | 0.556 | 0.944 | 0.393 | 2.020 | 0.184 |
| | | [0.807; | [0.592; | | | | | [1.653; | [0.079; |
| | | 0.933] | 0.773] | | | | | 2.470] | 0.427] |
| PTX3_PCT_LBP | 202 | 0.817 | 0.645 | 0.898 | 0.582 | 0.947 | 0.407 | 2.147 | 0.175 |
| | | [0.749; | [0.583; | | | | | [1.741; | [0.076; |
| | | 0.885] | 0.763] | | | | | 2.647] | 0.407] |
| PTX3_sCD14_LBP | 202 | 0.816 | 0.642 | 0.898 | 0.536 | 0.943 | 0.383 | 1.935 | 0.190 |
| | | [0.746; | [0.562; | | | | | [1.593; | [0.082; |
| | | 0.886] | 0.757] | | | | | 2.351] | 0.443] |
| PTX3_IP10_{—} | 199 | 0.800 | 0.640 | 0.898 | 0.447 | 0.931 | 0.346 | 1.623 | 0.228 |
| Calprotectin | | [0.728; | [0.583; | | | | | [1.366; | [0.098; |
| | | 0.871] | 0.733] | | | | | 1.927] | 0.534] |
| PTX3_LBP_{—} | 199 | 0.815 | 0.638 | 0.898 | 0.593 | 0.947 | 0.419 | 2.208 | 0.172 |
| Calprotectin | | [0.746; | [0.559; | | | | | [1.781; | [0.074; |
| | | 0.884] | 0.766] | | | | | 2.738] | 0.399] |
| PTX3_LBP_{—} | 199 | 0.811 | 0.635 | 0.898 | 0.513 | 0.939 | 0.376 | 1.845 | 0.199 |
| Gelsolin | | [0.741; | [0.559; | | | | | [1.527; | [0.085 |
| | | 0.881] | 0.747] | | | | | 2.230] | 0.463] |
| PTX3_Calprotectin_{—} | 199 | 0.759 | 0.634 | 0.898 | 0.453 | 0.932 | 0.349 | 1.643 | 0.225 |
| Gelsolin | | [0.685; | [0.562; | | | | | [1.381; | [0.096; |
| | | 0.832] | 0.726] | | | | | 1.954] | 0.526] |
| PTX3_PCT_{—} | 199 | 0.756 | 0.624 | 0.898 | 0.493 | 0.937 | 0.367 | 1.772 | 0.207 |
| Gelsolin | | [0.681; | [0.563; | | | | | [1.474; | [0.089; |
| | | 0.831] | 0.729] | | | | | 2.130] | 0.482] |
| PTX3_IP10_sCD14 | 202 | 0.811 | 0.623 | 0.898 | 0.503 | 0.939 | 0.367 | 1.808 | 0.203 |
| | | [0.738; | [0.541; | | | | | [1.502; | [0.087; |
| | | 0.883] | 0.734] | | | | | 2.176] | 0.472] |
| PTX3_PCT_sCD14 | 202 | 0.804 | 0.621 | 0.898 | 0.392 | 0.923 | 0.321 | 1.477 | 0.260 |
| | | [0.733; | [0.536; | | | | | [1.261; | [0.111; |
| | | 0.876] | 0.746] | | | | | 1.731] | 0.611] |
| PTX3_sCD14_{—} | 199 | 0.790 | 0.621 | 0.898 | 0.460 | 0.932 | 0.352 | 1.663 | 0.222 |
| Gelsolin | | [0.716; | [0.549; | | | | | [1.396; | [0.095; |
| | | 0.864] | 0.715] | | | | | 1.981] | 0.518] |
| PTX3_sCD14_{—} | 199 | 0.790 | 0.619 | 0.898 | 0.420 | 0.926 | 0.336 | 1.548 | 0.243 |
| Calprotectin | | [0.717; | [0.562; | | | | | [1.312; | [0.104; |
| | | 0.863] | 0.720] | | | | | 1.827] | 0.569] |
| PTX3_IP10_LBP | 202 | 0.832 | 0.609 | 0.898 | 0.438 | 0.931 | 0.338 | 1.598 | 0.233 |
| | | [0.760; | [0.534; | | | | | [1.349; | [0.100; |
| | | 0.903] | 0.742] | | | | | 1.891] | 0.545] |

The performance levels thus confirm that the PTX3 biomarkers can advantageously be combined with other biomarkers in the context of the identification of late-onset neonatal sepsis in newborns.

### 7.3 Combination of Biomarkers for Determining the Stopping of the Antibiotic Treatment

TABLE 9

| | Patients treated with antibiotics following suspicion of late-onset | |
|---|---|---|
| | neonatal sepsis = YES | |
| | Patients classified as “confirmed | Patients classified as “confirmed |
| | infection”^{b} and reclassified as | absence of infection” and |
| | not having an infection | reclassified as not having |
| Model selected | by the model (%)^{c} | an infection by the model (%)^{d} |
| PTX3/NGAL^{a} | 5/49 (10.2%) | 27/42 (64.3%) |
| PTX3/NGAL/Gelsolin^{a} | 5/49 (10.2%) | 23/42 (56.1%) |
| IL6^{a} | 5/51 (9.8%) | 10/42 (23.8%) |

(a) Models based on a logistic regression; (b) By the experts of the adjudication committee; (c) Denominator<51 in certain cases for the models based on a logistic regression because of two patients for whom data on the biomarkers were missing. (d) Denominator<42 in certain cases because of a patient for whom data on the biomarkers were missing.

Thus, among the patients who had received antibiotic therapy, five of them identified as having an infection by the adjudication committee were reclassified by the models as not having an infection, but this result was expected because the sensitivity of the model is fixed at 0.9.

However, it is noted that more than half (64.3% and 56.1%, respectively) of antibiotic therapies could have been avoided using the combination of the biomarkers PTX3/NGAL or PTX3/NGAL/gelsolin.

Conversely, the biomarker IL6, which had the best results among all of the biomarkers evaluated in terms of area under the ROC curve (AUC 0.864; Se 0.90; Sp 5.536 [0.799; 0.929]), does not make it possible to avoid a sufficient number of antibiotic therapies (only 23.8%) to be used and have a benefit for patients, thus confirming the surprising character obtained with the biomarker PTX3.

The PTX3 biomarker according to the invention, used in combination, therefore also finds a very particular application in patients in the neonatal unit as a tool in the early stopping of antibiotic treatments initially prescribed following suspicion of late-onset neonatal sepsis.

### Free Text of the Sequence Listing

In the present application, reference is made to the sequence listing(s) the identifier(s) (or “SEQ ID NO”) of which are listed below.

SEQ ID NO: 1 (PTX-3):
ENSDDYDLMYVNLDNEIDNGLHPTEDPTPCACGQEHSEWDKLFIMLE
NSQMRERMLLQATDDVLRGELQRLREELGRLAESLARPCAPGAPAEA
RLTSALDELLQATRDAGRRLARMEGAEAQRPEEAGRALAAVLEELRQ
TRADLHAVQGWAARSWLPAGCETAILFPMRSKKIFGSVHPVRPMRLE
SFSACIWVKATDVLNKTILFSYGTKRNPYEIQLYLSYQSIVFVVGGE
ENKLVAEAMVSLGRWTHLCGTWNSEEGLTSLWVNGELAATTVEMATG
HIVPEGGILQIGQEKNGCCVGGGFDETLAFSGRLTGFNIWDSVLSNE
EIRETGGAESCHIRGNIVGWGVTEIQPHGGAQYVS

Regardless of the form in which the listings are provided, these listings form part of the present application.

1. An in vitro method for determining whether a treatment with an antibiotic is able to be stopped in a newborn patient who possibly has late-onset neonatal sepsis and has previously received treatment with the antibiotic, said method comprising determining a protein concentration of PTX-3 in a biological sample previously obtained from said patient and comparing the concentration thus determined with a reference threshold value, wherein if the protein concentration of PTX-3 is lower than said reference threshold value then this indicates that the treatment with the antibiotic is able to be stopped and that the patient does not have late-onset neonatal sepsis.

2. The method of claim 1, wherein the protein concentration of PTX-3 is determined by an immunoenzymatic ELISA method.

3. The method of claim 1, wherein said biological sample is a biological blood, plasma or serum sample.

4. The method of claim 3, wherein the volume of the biological sample previously obtained from the patient is less than 50 μL.

5. The method of claim 1, wherein the reference threshold value for the protein concentration of PTX-3 is less than 3000 pg/mL.

6. The method of claim 1, further comprising determining the concentration of at least one other protein chosen from: IL-6, IL-10, PCT, IP10, sCD14, LBP, NGAL, calprotectin and gelsolin, wherein concentrations of the protein PTX-3 and of at least one of said other proteins lower than corresponding reference threshold values indicate that the treatment with the antibiotic is able to be stopped and that the patient does not have late-onset neonatal sepsis.

7. The method of claim 6, further comprising determining concentrations of the protein NGAL, wherein concentrations of the protein PTX-3 and NGAL lower than corresponding reference threshold values indicate that the treatment with the antibiotic is able to be stopped and that the patient does not have late-onset neonatal sepsis.

8. The method of claim 7, wherein the reference threshold value for the concentration of the protein NGAL is less than 300 ng/mL.

9. The method of claim 6, further comprising determining concentrations of the proteins NGAL and gelsolin, wherein concentrations of the protein PTX-3, NGAL and gelsolin lower than corresponding reference threshold values indicate that the treatment with the antibiotic is able to be stopped and that the patient does not have late-onset neonatal sepsis.

10. The method of claim 9, wherein the reference threshold value for the concentration of gelsolin is less than 300 μg/mL.

11. The method of claim 1, wherein only the concentration of PTX-3 is determined.

12. An in vitro method for determining whether a newborn patient has late-onset neonatal sepsis,
said method comprising determining a protein concentration of PTX-3 in a biological sample previously obtained from said patient and comparing the protein concentration of PTX-3 thus determined with a PTX-3 reference threshold value, wherein the protein concentration of PTX-3 being greater than the PTX-3 reference threshold value indicates that the patient has late-onset neonatal sepsis.

13. The method of claim 12, wherein the newborn patient previously evaluated as having late-onset neonatal sepsis is administered an antibiotic, wherein said antibiotic is in a form suitable for administration to said newborn patient.

14. A kit comprising reagents making it possible to determine the protein concentration of PTX3 and optionally of at least one other protein chosen from NGAL, IL-10, IL-6, calprotectin, LBP, PCT, sCD14, IP10 and gelsolin.

15. A method for determining whether a treatment with an antibiotic is able to be stopped in a newborn patient who possibly has late-onset neonatal sepsis and has previously received treatment with the antibiotic, wherein the method comprises providing the kit of claim 14.

16. The method of claim 12, further comprising determining a protein concentration of at least one other protein chosen from: IL-6, IL-10, PCT, IP10, sCD14, LBP, NGAL, calprotectin and gelsolin and comparing the protein concentration(s) thus determined with one or more corresponding reference threshold values.
